(19) United States
(12) Patent Application Publication
(10) Pub. No.: US 2013/0233327 A1
(43) Pub. Date: Sep. 12, 2013

(54) Hiccup Relief Device and Method

(71) Applicant: Charles A. RAY, San Diego, CA (US)

(72) Inventor: Charles A. RAY, San Diego, CA (US)

(21) Appl. No.: 13/870,566

(22) Filed: Apr. 25, 2013

Related U.S. Application Data
(63) Continuation of application No. 12/883,122, filed on Sep. 15, 2010, now abandoned.

(51) Int. Cl. A61B 19/00

(52) U.S. Cl. CPC A61B 19/00
USPC 128/898; 128/897

(57) ABSTRACT

The invention relates to a device for hiccup relief. The present invention provides a hiccup relief device having a main body having a substantially uniform longitudinal shape and a tab disposed on the main body, wherein the tab extends away from the body. The present invention also provides a hiccup relief method including: obtaining a hiccup relief device having a disrupting portion; positioning the hiccup relief device in a mouth of a user; and, delivering a consumable liquid over the hiccup relief device, wherein the disrupting portion disrupts a flow of liquid over the hiccup relief device.

### CROSS REFERENCE TO RELATED APPLICATION

This application is a nonprovisional application of U.S. application Ser. No. 12/883,122 filed on Sep. 15, 2010, and this application claims priority to that application and that application is incorporated in its entirety herein by reference.

### BACKGROUND OF THE INVENTION

1. Field of Invention

The present invention relates generally to the hiccup relief devices and methods. Particularly, the present invention relates to a device and method for use with the mouth of a user having hiccups, wherein the device and method provide hiccup relief.

2. Description of Related Art

Hiccups are a condition that effects everyone. Hiccups have been known to effect people for minutes or up to hours or even days. Hiccups are at the least annoying and can be at times debilitating. Very little is known about the exact cause of hiccups.

There is no conventional complete cure for hiccups. However, there are many conventional folk remedies. The folk remedies include many water drinking remedies, many startling-type remedies, and many breathing remedies. These remedies help to relieve some people some times, but as a whole are known to be relatively unreliable.

Some conventional devices attempt to solve the problem by creating a bulky cup with depression device. These devices fall short of solving the problem because they are still relatively unreliable, but also inconvenient.

Other conventional methods include the administration of pharmacological agents. Drugs that can be prescribed or administered for the treatment of hiccups include chlorpromazine, haloperidol, amphetamine, as well as some narcotics and herbal treatments. Due to the intensity of most of these drugs these remedies can only be used for extreme attacks and cannot be used for frequently reoccurring attacks.

Other conventional devices and methods include those used as cooling treatments. These include devices and methods for cooling both the outside of the neck and inside of the throat.

Still other devices and methods include those used to disrupt phrenic nerve transmission. These include tapping on a specific vertebrae, electrical stimulation, anesthetic injection, and surgical division. Although theoretically these treatments should be effective even the surgical division has been found to be unreliable.

The large number and diverse types of treatment show that there is no single effective remedy for curing hiccups.

Therefore, there remains a need for a simple, convenient, safe and effective treatment for hiccups.

### SUMMARY OF THE INVENTION

It is an object of the present invention to overcome the drawbacks and shortcomings of conventional hiccup relief devices and methods. Particularly, the present invention provides a hiccup relief device that includes an elongated body that includes a liquid flow disruption device. The device of the present invention is used by placing the device in the mouth and drinking a liquid over the device.

The present invention provides a device and method that is a simple hiccup treatment. The treatment includes simply placing the device in your mouth and drinking a liquid. There are no complicated steps or confusing directions. One person can easily perform the treatment on themselves without the aid of an assistant or medical professional.

Further, the present invention provides a device and method that is a convenient hiccup treatment. The device is small enough to fit into a purse, pocket, or kitchen or bathroom drawer. Therefore, a family can have the convenience of placing the device in an accessible drawer that any family member can access and use quickly and easily. Also, a person with chronic periodic bouts of hiccups can easily place the device in his pocket so that it is conveniently accessible anytime and anywhere a liquid is accessible.

Still further, the present invention, provides a device and method for a safe hiccup treatment. Although small and compact the device is not a choking hazard. The device does not involve any drugs or medical treatments that could be potentially hazardous. The method does not involve any scare tactics that could cause agitation of the individual.

Additionally, the present invention provides a device and method for an effective hiccup treatment. Unlike other water drinking treatments, the device includes both a mouth placement device and liquid agitator. The combination of placing the device in the mouth, biting down on the device, and the agitator creating a specific mix of liquid and air to flow down the throat makes for an effective remedy for curing hiccups.

This invention overcomes the drawbacks and shortcomings of the prior art conventional devices and systems.

The present invention provides a hiccup relief device comprising a main body having a substantially uniform longitudinal shape and a tab disposed on the main body, wherein the tab extends away from the body.

A hiccup relief device made in accordance with various exemplary embodiments of the present invention, are provided such that when the device is in use a portion of the device is operably configured to be disposed in a mouth of a user, wherein the tab is operably configured to be disposed above a tongue of the user and to be disposed further in the mouth than the main body.

The invention further provides a hiccup relief device including a body having a length, the length being of a dimension to span the jaw of the user, and a disruption member disposed on and extending from the body.

Still further, the present invention provides a hiccup relief method comprising obtaining a hiccup relief device having a disrupting portion, positioning the hiccup relief device in a mouth of a user, and delivering a consumable liquid over, beneath, around and through the hiccup relief device, wherein the disrupting portion disrupts a flow of liquid over the hiccup relief device.

These and other features and advantages of this invention are described in, or are apparent from, the following detailed description of various exemplary embodiments of the devices and methods according to this invention.

### BRIEF DESCRIPTION OF THE DRAWINGS

Various exemplary embodiments of this invention will be described in detail, with reference to the following figures, wherein;

FIG. 1 is a perspective view of a user utilizing a hiccup relief device made in accordance with this invention;

FIG. 2 is a top perspective view of a hiccup relief device made in accordance with this invention;

FIG. 3 is a bottom perspective view of the device of FIG. 2;

FIG. 4 is an right end view of the device of FIG. 2;

FIG. 5 is a top view of the device of FIG. 2;

FIG. 6 is an side view of the device of FIG. 2;

FIG. 7 is a partial cross-sectional diagram of the device of FIG. 2 in use;

FIG. 8 is a top view of an alternative embodiment of a hiccup relief device made in accordance with the present invention;

FIG. 9 is a top view of another alternative embodiment of a hiccup relief device made in accordance with the present invention;

FIG. 10 is a top view of yet another alternative embodiment of a hiccup relief device made in accordance with the present invention; and

FIG. 11 is a top view of another alternative embodiment of a hiccup relief device made in accordance with the present invention.

### DETAILED DESCRIPTION

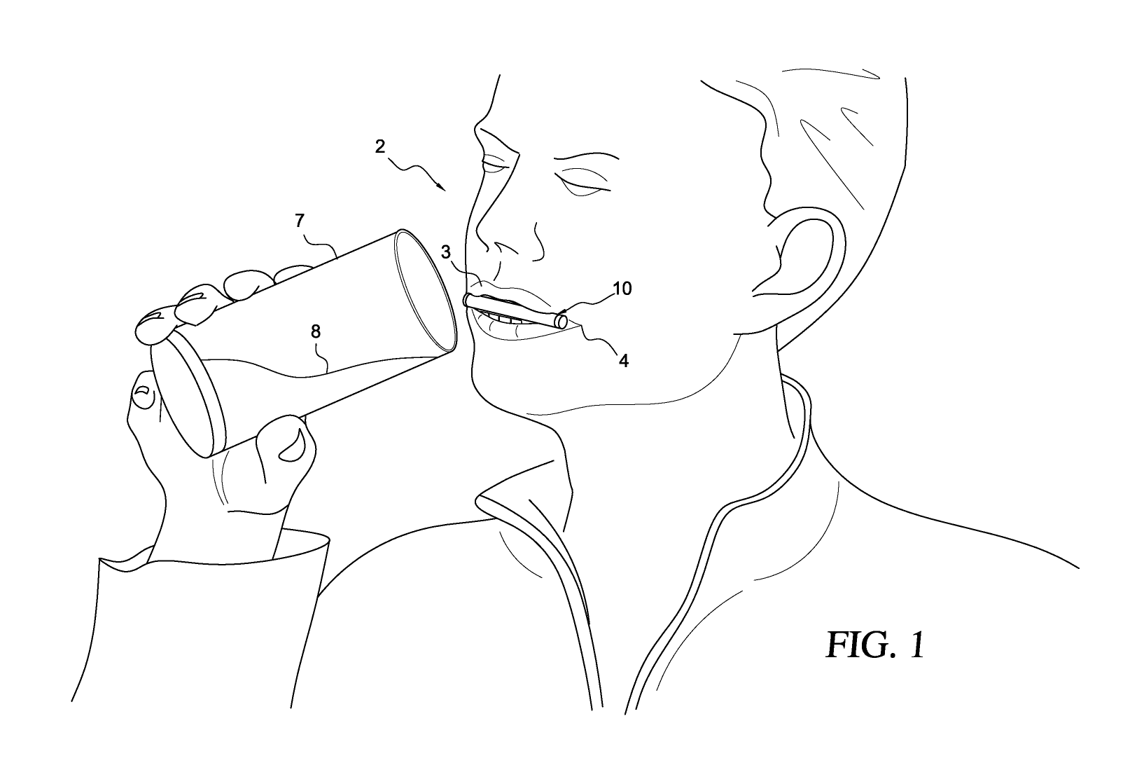

FIG. 1 is a perspective view of a user 2 with a hiccup relief device 10 made in accordance with the present invention. The device 10 preferably has an elongated shape. The device 10 is operably configured to be placed in a users mouth when in use. Particularly the device is operable configured to traverse the mouth and rests between the upper and lower jaws when in use. The use and operation of the device will be explained and detailed further below.

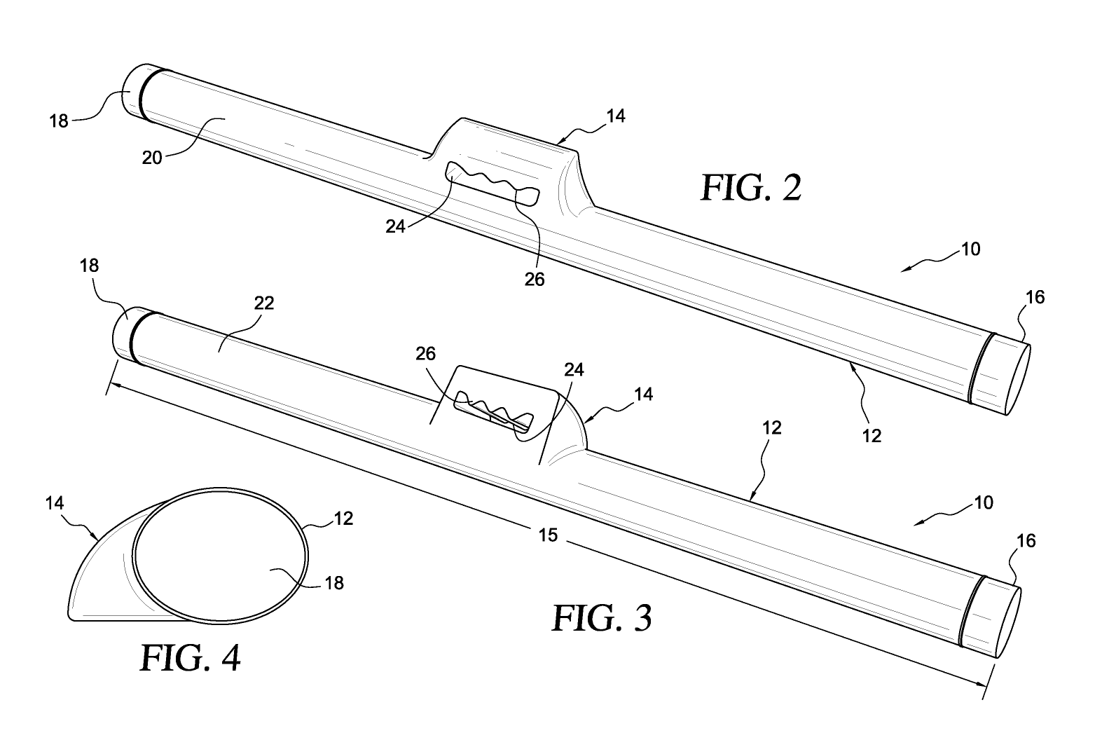

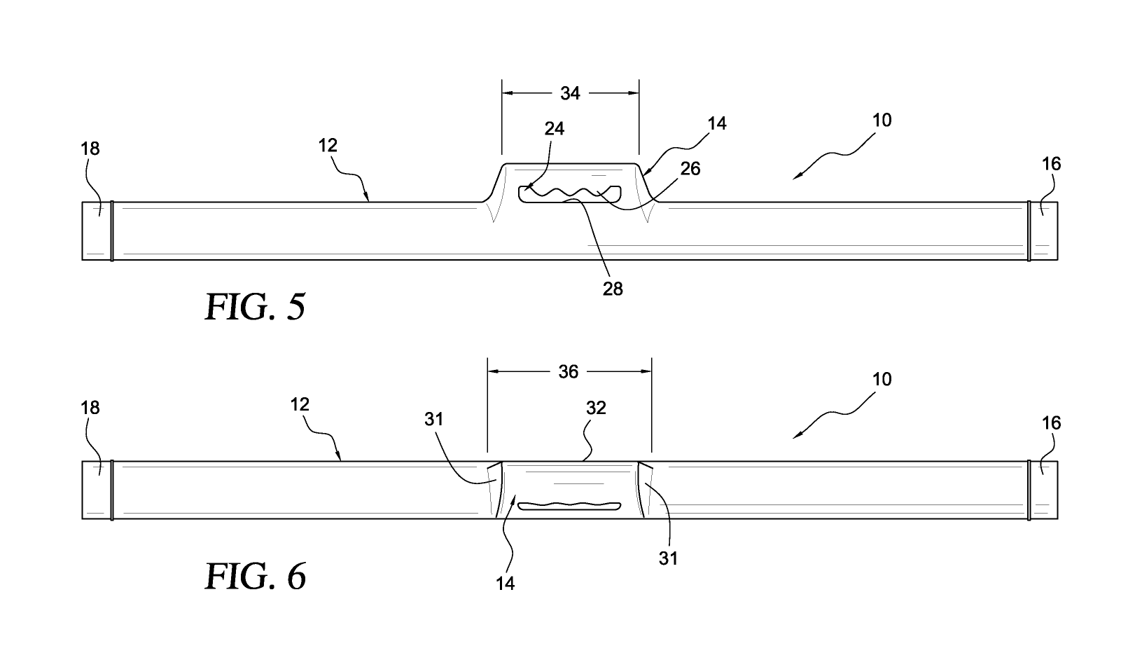

The device 10 includes a main body 12 and a tab 14, as shown in FIGS. 2-6. The tab 14 is disposed along on the main body 12. The main body includes a length 15 disposed between left and right ends 16 and 18. The length 15 is at least of a dimension to span the jaw of the user. The device 10 at a minimum rests upon the teeth of a user when in use as will be discussed further below. The tab 14 is preferably disposed near the center of the length 15. It should be appreciated that the tab could, in other exemplary embodiments, be disposed at a different location along the length of the main body.

In this exemplary embodiment, the tab 14 is integral with the main body 12. It should be appreciated that in other exemplary embodiments, the tab may be removably attached to the main body, or welded to the body, etc.

The main body 12 has a generally elliptical cross-sectional shape. However it should be appreciated that in other exemplary embodiments, the cross-sectional shape of the main body can be other shapes such as, but not limited to, circular, rectangular, hexagonal, etc.

The main body 12 includes a top surface 20 and a bottom surface 22. Since in this exemplary embodiment, the main body has an elliptical shape, the top surface and bottom surface 22 meet to form a contiguous surface.

The tab 14 extends from the top and bottom surfaces 20 and 22 of the main body 12. The tab 14 includes an orifice 24 and an orifice aft edge 26. The orifice is optional in other various exemplary embodiments. The tab 14 also includes a leading edge 28. The orifice 24 has a width, defined by the distance between the leading and aft edges 28 and 26. The tab further includes sides 31. The sides extend from the main body 12. The sides 31 are integral with the main body 12 in this embodiment. The sides 31 are preferred to be generally perpendicular to the main body 12. At the junction of the main body 12 and the sides 31, the connection is preferred to be rounded, creating a smooth transition between the main body 12 and the tab 14. The sides 31 are further preferred to be angled at about 50-90 degrees relative to the main body 12 toward the opposing side of the tab. Still further, the sides 31 are preferred to be angled at about 65-80 degrees relative to the main body 12 toward the opposing side of the tab.

The aft edge 26 is preferably non-planar. The profile or shape that the aft edge 26 forms can be seen in FIGS. 2-6. In this exemplary embodiment, the shape of the aft edge is that of a wave. It is preferred that the aft edge in this embodiment have a sinusoidal shape. The shape preferable spans the width of the orifice. It should be appreciated that in other exemplary embodiments, the shape does not extend completely along the width. Still further, those other shapes are employed and combined in other exemplary embodiments. The leading edge 28 preferable is planar with a linear shape.

It should be further appreciated that in other exemplary embodiments, multiple tabs may be disposed along the length of the main body as will be discussed further herein. The tab 14 is considered to be a flow disruption device.

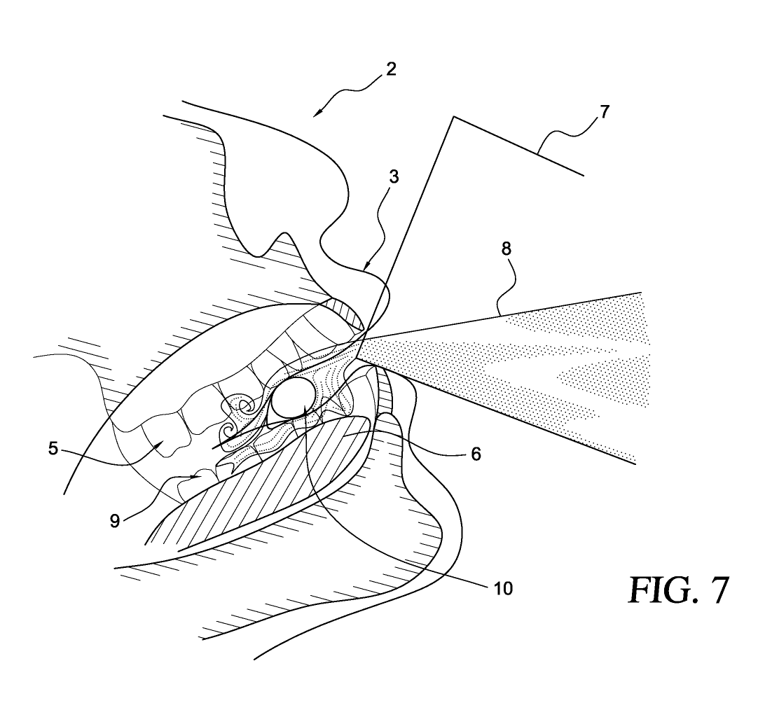

FIG. 7 and FIG. 1 display a hiccup relief method in accordance with the present invention. The method includes a variety of steps, including but not limited to positioning a hiccup relief device, pouring a liquid over the hiccup relief device and disrupting the flow of liquid. FIG. 7 shows an embodiment of a method of the present invention utilizing the device 10 and reference to the features of that device will be utilized in describing the features of the method. It should be appreciated that other devices could be utilized.

As shown in FIG. 7, when a user 2 desires hiccup relief, the user 2 positions the device 10 in its mouth 3. Preferably the device 10 is placed lengthwise across the mouth 3, between the upper and lower sets of teeth 5 and 9, such that the ends 16 and 18 of the device 10 extend out the sides of the mouth 3. The device 10 is further preferred to be placed back in the mouth, and more preferably adjacent to the junction 4 (see FIG. 1) of the lower and upper lips of the user 2. The device 10 is placed above the tongue 6 of the user 2.

The device 10 is preferably placed in the mouth 3 such that the tab 14 is positioned behind (more in the mouth) than the front of the main body 12, as shown in FIG. 7. The top surface 20 of the main body 12 is preferably facing or being directed toward the roof of the mouth. The user 2 closes his/her mouth on the device 10 to hold the device in place for use. The user slightly bites down on the device 10. The user does not need to grind down on the device 10 or bite down significantly to hold the device 10 in place.

An optional step of a method of the present invention includes slightly tilting the user's head back. The tilting of the head usually occurs naturally when drinking This step can occur prior to or after the position of the device.

Once in position, liquid 8 is poured over the device 10 in the mouth 3. The liquid 8 from a source 7 can be any consumable liquid. The source is preferably a drinking container. It should be appreciated that the source could be other things, such as a hose, straw, funnel or the like. Pouring the liquid in the mouth includes pouring the liquid in the mouth 3 and over the device 10. As the user 2 swallows, the liquid will tend to flow through the mouth and down the throat of the user. During that travel, the liquid will flow around the device 10.

Disrupting the liquid being poured over the device is another step in the method of the present invention. As the liquid is flowing around the device in the mouth, the tab 14 is operably configured to disrupt the uniform flow of the liquid around the device. The disruption occurs because of the tab extends away from the device forcing some of the liquid to flow at a different rate than other liquid flowing over the remainder of the device. Further for the devices incorporating an orifice in the disruption tab in accordance with the present invention, some of the liquid falls into and through the orifice. This structure enables air pockets to be created in the flow of liquid further enhancing the effectiveness of the hiccup relief of the device and method of the present invention. Still further, with the device 10 having an orifice with a profiled aft edge, the flow of the liquid is even further disrupted as it passes over the device and through the orifice.

Holding the device of the present invention in the mouth tenses the jaw and throat muscles and that in combination with the cavitation created by liquid flowing over the device offers hiccup relief. The tensing of the jaw on a device made in accordance with the present invention also excites and/or contracts the diaphragm, which aids in hiccup relief

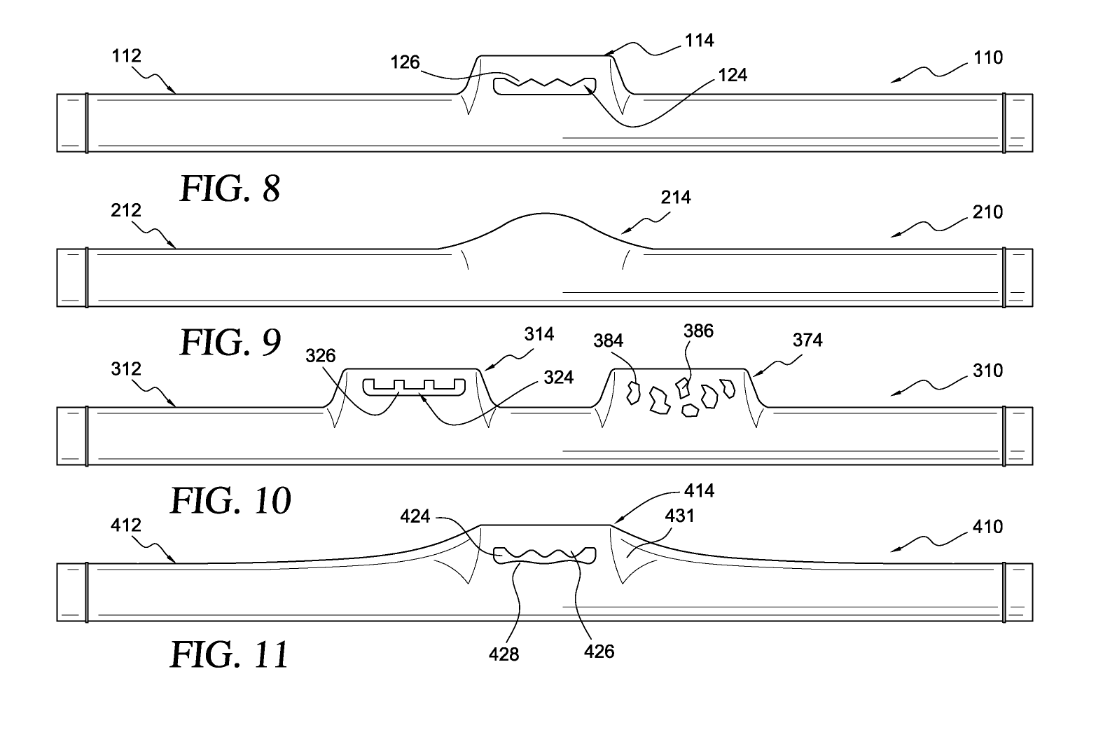

FIGS. 8, 9, 10 and 11 display top views of devices 110, 210, 310 and 410, respectively, all of which are alternative embodiments of a hiccup relief device made in accordance with the present invention. These devices 110, 210, 310 and 410 are similar to the device 10 described above. Each of the devices have a main body member (112, 212, 312 and 412 respectively) and a tab (114, 214, 314 and 414 respectively). The devices 110, 210, 310 and 410 are the same as the device 10 and include all of the features of the device 10.

The device 110 is different from the device 10 in that the aft edge 126 of the orifice 124 of the tab 114 has a different shaped profile, as shown in FIG. 8. In this exemplary embodiment, the edge includes sharper transitions in the curves and is less sinusoidal. This creates a saw-tooth look from the top view. The change in the aft edge profile impacts the flow of the liquid over the device when in use.

The device 210 is different from the device 10 in that the tab 214 does not have an orifice. Further the tab 214 has a rounded profile as shown in FIG. 9.

The device 310 is different from the device 10 in that it includes more than one disruption tab. In this embodiment, there are two disruption tabs 314 and 374. Both tabs include similar features to the tab 14. It should be appreciated that in other exemplary embodiments devices can include additional tabs. In this embodiment, tabs 374 and 314 are substantially the same size. It should be appreciated, that the tabs could be different sizes in other embodiments. The tab 374 includes multiple orifices 384. At least one of the orifices includes a profiled or shaped aft edge 386 as shown in FIG. 10. The change in the profiles of the aft edges and the number of tabs impact the flow of the liquid over the device when in use. Aft edge 326 of the orifice 324 of the tab 314 has a different shaped profile, as shown in FIG. 9. In this exemplary embodiment, the edge includes straight lengths with sharper transitions and plateaus. This creates a tooth look from the top view. The change in the aft edge profile impacts the flow of the liquid over the device when in use.

The device 410 is different from the device 10 in that the sides 431 of the tab 414 are less than 45 degrees from the main body 412. Further the sides 431 are more rounded. In this embodiment the tab 414 is more integral or continuous with the main body. The tab in this embodiment is a bump or protrusion disposed along the length of the main body 412. Further, the leading edge 428 of the orifice 424 of the tab 414, is also profiled in this embodiment as opposed to being planer shaped. In this embodiment, the edge includes a slight sinusoidal curve. It should be appreciated that other shapes and combination of shapes can be used for either the leading edges or the aft edges.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

What is claimed is:

1. A hiccup relief device comprising:
a main body having a substantially uniform longitudinal shape; and,
a tab disposed on the main body, wherein the tab extends away from the body.

2. The hiccup relief device, as recited in claim 1, wherein the tab includes an orifice.

3. The hiccup relief device, as recited in claim 1, wherein the tab is integral with the main body.

4. The hiccup relief device, as recited in claim 1, wherein the tab is operable configured

5. The hiccup relief device, as recited in claim 1, wherein the tab increases the width of the device along an area wherein the tab is disposed

6. The hiccup relief device, as recited in claim 1, wherein the device is operably configured to be placed in a mouth of a user wherein the tab is disposed above a tongue of the user.

7. The hiccup relief device, as recited in claim 2, wherein the orifice extends all the way through the tab.

8. The hiccup relief device, as recited in claim 1, wherein the tab includes a plurality of orifices.

9. The hiccup relief device, as recited in claim 1, wherein the tab includes an orifice having an aft edge and the aft edge has a generally sinusoidal shape along a length of the aft edge.

10. The hiccup relief device, as recited in claim 1, wherein the tab includes an orifice having an aft edge and the aft edge has a generally non-sinusoidal shaped length.

11. The hiccup relief device, as recited in claim 1, wherein the main body has first and second ends and at least one of the first and second ends have a generally elliptical cross-sectional shape.

12. The hiccup relief device, as recited in claim 1, wherein the tab includes sides, and the sides tab sides are sloped toward a center of tab

13. The hiccup relief device, as recited in claim 1, wherein the tab in integral with the main body.

14. The hiccup relief device, as recited in claim 1, wherein tab has a generally rounded shape with one continuous rounded side, the rounded side begins and ends on the main body.

15. The hiccup relief device, as recited in claim 1, wherein when the device is in use a portion of the device is operably configured to be disposed in a mouth of a user, wherein the tab is operably configured to be disposed above a tongue of the user and to be disposed further in the mouth than the main body.

16. A hiccup relief device comprising:
a body having a length, the length being of a dimension to span the jaw of the user; and, a disruption member disposed on and extending from the body.

17. The hiccup relief device of claim 16, wherein the disruption member includes at least one orifice.

18. The hiccup relief device of claim 16, wherein the device includes a plurality of disruption members, and wherein at least one of the plurality of disruption members includes an orifice.

19. A hiccup relief method comprising:
obtaining a hiccup relief device having a disrupting portion;
positioning the hiccup relief device in a mouth of a user; and,
delivering a consumable liquid over the hiccup relief device, wherein the disrupting portion disrupts a flow of liquid over the hiccup relief device.

20. The hiccup relief method of claim 19, wherein the positioning includes placing the device adjacent canine teeth of a user and closing a jaw of the user on the device.
